(19) United States
(12) Patent Application Publication
(10) Pub. No.: US 2026/0114814 A1
(43) Pub. Date: Apr. 30, 2026

(54) APPARATUS AND METHOD FOR DIAGNOSING AUTISM SPECTRUM DISORDER(ASD) USING MULTI-HEAD ATTENTION-BASED DYNAMIC FUNCTIONAL CONNECTIVITY

(71) Applicant: UIF (UNIVERSITY INDUSTRY FOUNDATION), YONSEI UNIVERSITY, Seoul (KR)

(72) Inventors: SUNGBAE CHO, Seoul (KR); HYUNGJUN MOON, Seoul (KR)

(73) Assignee: UIF (UNIVERSITY INDUSTRY FOUNDATION), YONSEI UNIVERSITY, Seoul (KR)

(21) Appl. No.: 18/934,133

(22) Filed: Oct. 31, 2024

(30) Foreign Application Priority Data
Oct. 28, 2024 (KR) 10-2024-0149027

(51) Int. Cl. A61B 5/00; A61B 5/055; G16H 50/20

(52) U.S. Cl. CPC A61B 5/7264; A61B 5/0042; A61B 5/055; G16H 50/20

(57) ABSTRACT

An apparatus for diagnosing an autism spectrum disorder (ASD) based on a graph neural network includes a preprocessor configured to acquire brain image data, designate a region of interest (ROI) of the brain, and generate preprocessed data for neural network input, a spatial feature extractor configured to extract spatial features from the preprocessed data, a temporal feature extractor configured to analyze changes in brain activity over time and extract attention-based temporal features, a spatial and temporal convergence feature unit configured to analyze spatiotemporal correlations by combining the spatial features and the temporal features, a graph generator configured to convert connectivity between regions of interest into a graph structure and implement the same as nodes and edges, and a graph classifier configured to analyze a spatiotemporal pattern of the connectivity through the graph structure and classify whether or not there is an ASD.

### CROSS-REFERENCE TO RELATED APPLICATION

This application claims under 35 U.S.C. § 119 (a) the benefit of Korean Patent Application No. 10-2024-0149027 filed on Oct. 28, 2024, the entire contents of which is incorporated herein by reference.

### TECHNICAL FIELD

The present disclosure relates to an autism spectrum disorder (ASD) diagnosis technique, and more specifically, to an apparatus and method for diagnosing autism spectrum disorder using a graph neural network (GNN) which can improve the accuracy of a diagnosis of autism spectrum disorder by combining a multi-head attention technique and a graph neural network (GNN).

### BACKGROUND

Autism spectrum disorder (ASD) is a neurodevelopmental disorder exhibiting heterogeneous characteristics in patients, including variability in developmental progression and neuroanatomical features, making diagnosis a complex and challenging task. Recently, deep learning models based on functional magnetic resonance imaging (fMRI) have shown promising results, but existing studies have mainly focused on generalized global activation patterns, failing to sufficiently capture regional characteristics, and have had limitations in accurately assessing disease symptoms.

Methods utilizing functional connectivity (FC) are useful for modeling the functional relationships of the brain, but many existing methods focus only on structural characteristics or do not consider heterogeneous external characteristics of data, which reduces the efficiency of ASD diagnosis and overlooks temporary and regional characteristics. In addition, some symptoms may appear intensively only in certain time periods and regional areas, and thus it is important to effectively capture such characteristics.

Korean Patent Publication No. 10-2023-0024667 (2023.02.21) provides a 4D fMRI autism prediction and early diagnosis method and device that enables accurate prediction and early diagnosis of autism by providing a model that learns patient-specific spatial variation factors while minimizing information loss of time-series features and extracting autism characteristics through weighting of time-series connectivity. The autism prediction and diagnosis device outputs autism characteristics through an autism prediction and diagnosis model that includes a spatial structure model of a brain region and a dynamic time-series connectivity model of the brain region using time-series brain image data, thereby accurately predicting and diagnosing autism early.

### PRIOR ART LITERATURE

### Patent Literature

Korean Patent Publication No. 10⁻²⁰²³-0024667 (2023.02.21)

### DESCRIPTION

### Problem to be Solved

One embodiment of the present disclosure provides a graph neural network-based autism spectrum disorder (ASD) diagnosis apparatus and method capable of improving the accuracy of ASD diagnosis by combining a multi-head attention technique and a graph neural network (GNN).

One embodiment of the present disclosure provides a graph neural network-based autism spectrum disorder (ASD) diagnosis apparatus capable of obtaining brain image data, converting connectivity between regions of interest into a graph structure, analyzing spatiotemporal patterns of connectivity, and classifying whether or not there is an autism spectrum disorder (ASD).

One embodiment of the present disclosure provides a graph neural network-based autism spectrum disorder (ASD) diagnosis apparatus capable of extracting spatial features and attention-based temporal features of brain image data and analyzing spatiotemporal patterns to classify whether or not there is an autism spectrum disorder (ASD).

### Solution

In one embodiment, an apparatus for diagnosing an autism spectrum disorder (ASD) using a graph neural network includes a preprocessor configured to acquire brain image data, designate a region of interest (ROI) of the brain, and generate preprocessed data for neural network input, a spatial feature extractor configured to extract spatial features from the preprocessed data, a temporal feature extractor configured to analyze changes in brain activity over time and extract attention-based temporal features, a spatial and temporal convergence feature unit configured to analyze spatiotemporal correlations by combining the spatial features and the temporal features, a graph generator configured to represent connectivity between regions of interest as a graph structure and implement nodes and edges in the graph structure, and a graph classifier configured to analyze a spatiotemporal pattern of the connectivity through the graph structure to classify whether or not there is an ASD.

The preprocessor may acquire fMRI data as the brain image data and acquire the region of interest of the brain as a plurality of window unit images through a sliding window to generate the preprocessed data.

The spatial feature extractor may perform a CNN operation on the preprocessed data to generate a feature map representing a degree of activation or an activity pattern with respect to the region of interest as the spatial features.

The temporal feature extractor may apply a bidirectional long short-term memory (LSTM) network to the preprocessed data to generate temporal features of previous or subsequent activities occurring in the region of interest.

The temporal feature extractor may apply a multi-head attention mechanism to the temporal features to generate attention representing temporal interactions of the activities. The temporal feature extractor may integrate the temporal features and the attention and perform global average pooling to summarize the weighted changes in the brain activity into the attention-based temporal features.

The spatial and temporal convergence feature unit may generate a correlation matrix representing a degree of spatiotemporal interaction of the brain activity by combining the spatial features and the attention-based temporal features to represent the spatiotemporal correlations.

The graph generator may quantify connectivity between regions of interest of the brain through the correlation matrix derived by the spatial and temporal convergence feature unit to determine a weight of the edge.

The graph classifier may generate graph embedding for the graph structure and classify whether or not there is an ASD through a fully connected layer and a softmax layer.

In one embodiment, a method of diagnosing an ASD using a graph neural network, performed in an apparatus for diagnosing an ASD using a graph neural network includes a preprocessing step of acquiring brain image data, designating a region of interest (ROI) of the brain, and generating preprocessed data for neural network input, a spatial feature extraction step of extracting spatial features with respect to the region of interest of the brain from the preprocessed data, a temporal feature extraction step of extracting attention-based temporal features from the preprocessed data by deriving weighted changes in brain activity over time with respect to the region of interest of the brain, a spatial and temporal convergence feature step of analyzing spatiotemporal correlations of brain activity by combining the spatial features and the attention-based temporal features, a graph generation step of representing connectivity between regions of interest of the brain as a graph structure, each region of interest being implemented as a node and the connectivity being implemented as an edge in the graph structure, and a graph classification step of analyzing a spatiotemporal pattern of the connectivity through the graph structure to classify whether or not there is an ASD.

### Advantageous Effects

The disclosed technique has the following effects. However, it should be understood that the scope of the disclosed technique is not limited thereby since it does not mean that a specific embodiment must include all or only the following effects.

The autism spectrum disorder (ASD) diagnosis apparatus according to one embodiment of the present disclosure extracts spatial features and attention-based temporal features of brain image data by combining a multi-head attention technique and a graph neural network (GNN), and analyzes spatiotemporal interactions of the brain on the basis of the extracted features to accurately predict and diagnose ASD.

The ASD diagnosis apparatus according to one embodiment of the present disclosure obtains brain image data, converts connectivity between regions of interest into a graph structure, and analyzes spatiotemporal patterns of connectivity using a graph neural network to determine ASD, thereby improving the accuracy of diagnosis.

The ASD diagnosis apparatus according to one embodiment of the present disclosure can rapidly and accurately predict ASD through a graph neural network through convergence of spatial features and attention-based temporal features derived from brain imaging data and analysis of spatiotemporal patterns.

### BRIEF DESCRIPTION OF THE DRAWINGS

FIG. 1 is a flowchart illustrating an operation process of an ASD diagnosis apparatus using a graph neural network according to the present disclosure.

FIG. 2 is a diagram illustrating a configuration of the ASD diagnosis apparatus according to the present disclosure.

FIG. 3 is a flowchart illustrating the operation of the ASD diagnosis apparatus of FIG. 2.

FIG. 4 is a box plot visually comparing the accuracy of diagnosis of an ASD diagnosis method according to the present disclosure and various existing models.

FIG. 5 is a graph showing receiver operating characteristic (ROC) curves evaluating the performance of the ASD diagnosis apparatus according to the present disclosure.

FIG. 6 is a brain scan image visually showing results of activation of a region of interest (ROI) of the brain analyzed by the ASD diagnosis apparatus according to the present disclosure.

FIG. 7 is a diagram visually showing analysis results of the ASD diagnosis apparatus according to the present disclosure, and shows correlation matrices and brain regional connectivity graphs for comparison of functional connectivities in the brains of an ASD patient and a normal control group.

FIG. 8 is a diagram showing comparison of brain functional connectivities of an ASD patient and a normal control group as a result of analysis of the ASD diagnostic apparatus according to the present disclosure.

### DETAILED DESCRIPTION

Specific structural or functional descriptions in the embodiments of the present disclosure introduced in this specification or application are only for description of the embodiments of the present disclosure. The descriptions should not be construed as being limited to the embodiments described in the specification or application. The present disclosure may, however, be embodied in many different forms, but should be construed as covering modifications, equivalents or alternatives falling within ideas and technical scopes of the present disclosure. Further, since effects disclosed herein do not mean that a specific embodiment should include all or only the effects, the scope of the present disclosure should not be construed as being limited thereto.

Meanwhile, the meaning of terms described herein will be understood as follows.

It will be understood that, although the terms “first”, “second”, etc. may be used herein to distinguish one element from another element, these elements should not be limited by these terms. For instance, a first element discussed below could be termed a second element without departing from the teachings of the present disclosure. Similarly, the second element could also be termed the first element.

It will be understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present. Other expressions that explain the relationship between elements, such as “between”, “directly between”, “adjacent to” or “directly adjacent to” should be construed in the same way.

In the present disclosure, the singular forms are intended to include the plural forms as well, unless the context clearly indicates otherwise. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

In each step, reference characters (e.g. a, b, c, etc.) are used for the convenience of description. The reference characters do not designate the order of the steps, and the steps may be performed in a different order unless the context clearly indicates otherwise. That is, the steps may be performed in the specified order, may be performed substantially simultaneously, or may be performed in a reverse order.

The present disclosure can be implemented as a computer-readable code on a computer-readable recording medium. The computer-readable recording medium includes all types of recording devices in which data readable by a computer system is stored. Examples of the computer-readable recording medium include ROM, RAM, CD-ROM, magnetic tape, floppy disk, an optical data storage device, etc. In addition, the computer-readable recording medium may be distributed in a computer system connected via a network, so that computer-readable codes may be stored and executed in a distributed manner.

Unless otherwise defined, all terms including technical and scientific terms used herein have the same meaning as commonly understood by one of ordinary skill in the art to which the present disclosure belongs. It will be further understood that terms used herein should be interpreted as having a meaning that is consistent with their meaning in the context of this specification and the relevant art and will not be interpreted in an idealized or overly formal sense unless expressly so defined herein.

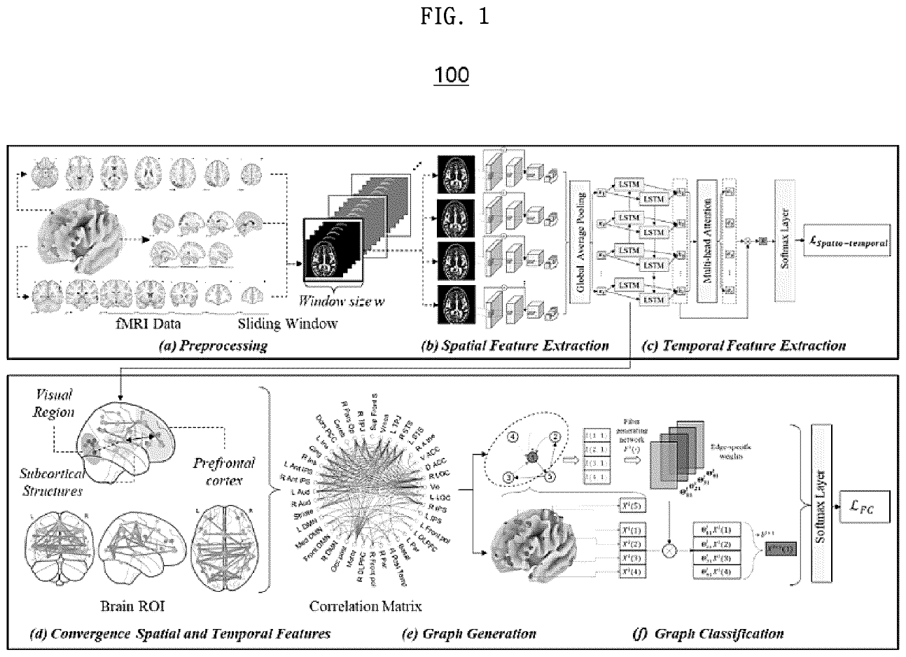

FIG. 1 is a flowchart illustrating an operation process of an autism spectrum disorder (ASD) diagnosis apparatus 100 using a graph neural network according to the present disclosure.

In FIG. 1, the ASD diagnosis apparatus 100 using a graph neural network performs (a) preprocessing, (b) spatial feature extraction, (c) temporal feature extraction, (d) convergence of spatial and temporal Features, (e) graph generation, and (f) graph classification.

### (a) Preprocessing

In the process (a) of FIG. 1, the ASD diagnosis apparatus 100 preprocesses brain image data. Here, the brain image data may be fMRI data, and the fMRI data is divided into 3D blocks at each time interval through a sliding window method, and preprocessed data can be generated through such blocks. The preprocessed data can be used as basic data for neural network input.

### (b) Spatial Feature Extraction

In the process (b) of FIG. 1, the ASD diagnosis apparatus 100 may extract spatial features of the brain on the basis of the preprocessed data. The ASD diagnosis apparatus 100 may use a convolutional neural network (CNN) to convert an activation pattern of each brain region into a feature map, and extract spatial features through the feature map.

### (c) Temporal Feature Extraction

In the process (c) of FIG. 1, the ASD diagnosis apparatus 100 may extract temporal features on the basis of the extracted spatial features. The ASD diagnosis apparatus 100 may learn changes in brain activity over time by applying a bidirectional long short-term memory (LSTM) network, and may extract temporal features by weighting temporal interactions through a multi-head attention mechanism.

### (d) Convergence of Spatial and Temporal Features

In the process (d) of FIG. 1, the ASD diagnosis apparatus 100 may analyze spatiotemporal correlations by combining the spatial features and temporal features. The combined spatial and temporal features of each brain region generate a correlation matrix, and the correlation matrix can numerically express spatiotemporal interactions between regions of interest in the brain. That is, the ASD diagnosis apparatus 100 can systematically analyze the correlation of brain activity.

### (e) Graph Generation

In the process (e) of FIG. 1, the ASD diagnosis apparatus 100 may convert connectivity between brain regions into a graph structure on the basis of the correlation matrix. The correlation matrix may be converted into an adjacency matrix of a graph, a brain region may be implemented as a node, and a correlation may be implemented as an edge. Here, a weight of each edge can reflect the strength of the correlation.

### (f) Graph Classification

In the process (f) of FIG. 1, the ASD diagnosis apparatus 100 may classify whether or not ASD is present on the basis of the generated graph. The ASD diagnosis apparatus 100 may analyze a spatiotemporal connection pattern of the brain using a graph neural network (GNN), and may finally classify whether or not ASD is present through a fully connected layer and a softmax layer.

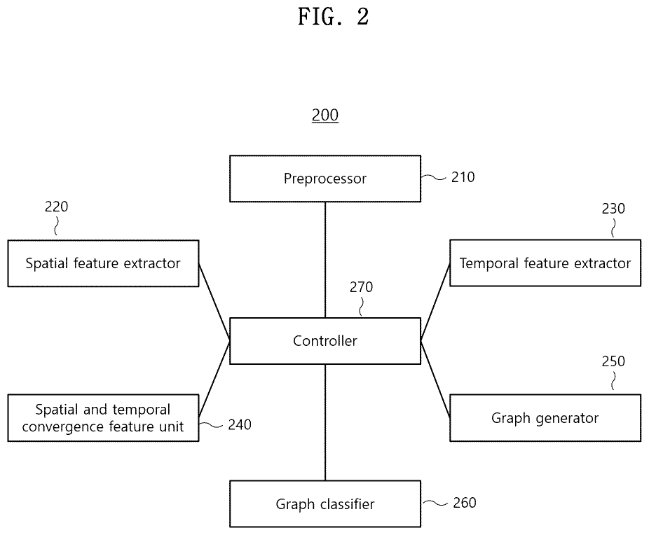

FIG. 1 visually represents the process of diagnosing an ASD by the ASD diagnosis apparatus 100 by utilizing the functional connectivity and spatiotemporal features of the brain. FIG. 2 is a diagram illustrating a configuration of the ASD diagnosis apparatus 200 according to the present disclosure.

Referring to FIG. 2, the ASD diagnosis apparatus 200 may include a preprocessor 210, a spatial feature extractor 220, a temporal feature extractor 230, a spatial and temporal convergence feature unit 240, a graph generator 250, a graph classifier 260, and a controller 270.

The preprocessor 210 may acquire brain image data, designate a region of interest (ROI) of the brain, and generate preprocessed data for neural network input. The preprocessor 210 generates multiple window unit images using a sliding window technique on the basis of fMRI data, and the generated preprocessed data may be used as input for subsequent neural network analysis. The preprocessor 210 provides data by which spatial and temporal characteristics of the brain can be analyzed, and may be utilized in the process of diagnosing ASD.

The spatial feature extractor 220 may extract spatial features with respect to a region of interest of the brain using preprocessed data. The spatial feature extractor 220 may perform a convolutional neural network (CNN) operation based on preprocessed fMRI data and generate a feature map representing the degree of activation or activity pattern in a specific region of the brain. The generated feature map defines the spatial features of the brain and may be used as main input data for ASD diagnosis.

In addition, the spatial feature extractor 220 may extract spatial features more precisely by applying a residual convolutional neural network (ResNet) structure. The residual neural network operates in a manner of adding a convolution operation F(x) to input data x as represented by the following mathematical expression 1, and accordingly, it can solve a gradient vanishing issue that may occur when a neural network becomes deeper.

$$H ( x ) = F ( x ) + x \qquad [ Mathematical expression 1 ]$$

Here, H(x) represents the residual operation, and F(x) is defined as a convolutional operation. The convolutional operation F(x) is computed based on a filter weight

$${w}_{ab}^{l}$$

and input data

$${x}_{( i + a ) ( j + b )}^{l - 1}$$

of the previous layer according to the following mathematical expression 2.

$$F ( x ) = {\sum }_{a = 0}^{m - 1} {\sum }_{b = 0}^{m - 1} {w}_{ab}^{l} {x}_{( i + a ) ( j + b )}^{l - 1} \qquad [ Mathematical expression 2 ]$$

This operation can play an important role in analyzing a regional activation state of the brain and modeling an activity pattern of a specific region of interest. Accordingly, it is possible to provide more precise spatial structural features of the brain for ASD diagnosis.

The spatial feature extractor 220 may analyze spatiotemporal interactions of brain activity by combining the generated feature map with a spatial and temporal convergence feature analyzer, and such data can be utilized as important basic data for increasing the accuracy of ASD diagnosis.

The temporal feature extractor 230 may analyze weighted changes of brain activity over time in the region of interest of the brain on the basis of preprocessed data and extract attention-based temporal features. The temporal feature extractor 230 may generate temporal features by analyzing previous and subsequent activities that have occurred in the region of interest by applying a bidirectional long short-term memory (LSTM) network, and may derive feature data reflecting temporal interactions of brain activity. The generated temporal features provide important input data for ASD diagnosis and can effectively reflect temporal changes and interactions of brain activity to improve the accuracy of ASD diagnosis.

The temporal feature extractor 230 may evaluate the relative importance of each temporal feature and assign weights to important temporal interactions of brain activities by applying a multi-head attention mechanism. An attention mechanism can analyze temporal features from various perspectives and emphasize important interactions of neural activities. In addition, the temporal features derived through multi-head attention can be summarized by performing global average pooling, and this can be utilized as input to a graph neural network (GNN) that predicts presence or absence of ASD.

More specifically, the temporal feature extractor 230 is a core component that analyzes and extracts temporal features in the ASD diagnosis apparatus, and can analyze temporal interactions by applying a bidirectional LSTM (BILSTM) network and the multi-head attention mechanism on the basis of preprocessed fMRI data.

The BiLSTM network may learn temporal dependencies for given temporal data. Temporal features obtained from fMRI data may be input into the bidirectional LSTM network, and thus temporal features that simultaneously reflect past and future brain activities can be extracted. For example, spatial features x₁, x₂, . . . , xₖ generated through a CNN operation can be input into the BiLSTM network, and the BiLSTM network can process this data to generate a series of hidden states h₁, h₂, . . . , h_{N}. These hidden states reflect temporal interactions and can be used as important temporal features for ASD diagnosis.

The multi-head attention mechanism can serve to compute the importance between temporal features. This mechanism can independently analyze temporal interactions through multiple heads and assign weights to important temporal features. In each head, an attention weight aᵢⱼ is computed on the basis of an interaction between hidden states hᵢ and hⱼ, which can be defined through a softmax function as represented by the following mathematical expression 3.

$${a}_{ij} = \frac{exp ( score ( {h}_{i} , {h}_{j} ) )}{{\sum }_{k} exp ( score ( {h}_{i} , {h}_{j} ) )} \qquad [ Mathematical expression 3 ]$$

Here, score (hᵢ, hⱼ) is a function for measuring compatibility between hidden states hᵢ and hⱼ and can be computed as represented by the following mathematical expression 4.

$$score ( {h}_{i} , {h}_{j} ) = {W}_{q} {h}_{i} \cdot {W}_{k} {h}_{j} \qquad [ Mathematical expression 4 ]$$

W_{q} and Wₖ can be defined as weight matrices for query and key in the attention mechanism. A final output feature rᵢ for each hidden state hᵢ is computed as the weighted sum of all hidden states and can be represented as the following mathematical expression 5.

$${r}_{i} = {\sum }_{j} {a}_{ij} {h}_{j} \qquad [ Mathematical expression 5 ]$$

This attention computation is repeated independently through multiple heads and is finally integrated into a single concatenated vector. Through this process, the multi-head attention mechanism can effectively analyze interactions of temporal features and extract important temporal correlations.

Finally, the extracted temporal features can be summarized through global average pooling, and the summarized features can be provided as input to a graph neural network (GNN) and used to predict presence or absence of ASD.

Therefore, the temporal feature extractor 230 can perform the function of analyzing temporal interactions using the BiLSTM network and the multi-head attention mechanism and precisely extracting temporal features necessary for ASD diagnosis.

The spatial and temporal convergence feature unit 240 may serve to analyze spatiotemporal correlations of brain activities by combining spatial features and attention-based temporal features. The spatial and temporal convergence feature unit 240 may combine the spatial features generated by the spatial feature extractor 220 and the attention-based temporal features generated by the temporal feature extractor 230 to generate a correlation matrix representing the degree of spatiotemporal interaction between brain activities.

The generated correlation matrix quantifies a spatiotemporal correlation between regions of interest in the brain and can be used as main analysis data for ASD diagnosis. The spatial and temporal convergence feature unit 240 may clearly ascertain spatiotemporal interactions between brain regions through this correlation matrix and use the same to input important features related to ASD into a graph neural network (GNN).

Specifically, the spatial and temporal convergence feature unit 240 includes a function of learning dynamic connectivity and can compute an interaction between brain regions on the basis of a connectivity matrix. Such a correlation matrix represents the connectivity between the i-th brain region and the j-th brain region and can be computed using the following mathematical expression 6.

$${C}_{ij} = f ( {r}_{i} , {r}_{j} ) \qquad [ Mathematical expression 6 ]$$

Here, Cᵢⱼ is a value representing the correlation between the i-th region and the j-th region of the brain, and rᵢ and rⱼ are feature representations derived from the respective brain regions. This correlation matrix reflects a dynamic relationship between brain regions on the basis of time-series data and can provide important information for ASD diagnosis.

In addition, the spatial and temporal convergence feature unit 240 may convert the connectivity matrix into a graph form to express each brain region as a node and a correlation as an edge. Through this GNN, it is possible to analyze an interaction between brain regions and derive key features required for ASD diagnosis. Through such dynamic connectivity analysis, the spatial and temporal convergence feature unit 240 can effectively analyze the complex neural network structure of the brain and functional characteristics through the neural network structure.

The mathematical expressions used in the embodiment of the present disclosure are based on the above-described connectivity matrix computation method and are applied at the time of modeling a correlation between different regions of the brain. For example, a spatiotemporal correlation matrix can be computed by the following mathematical expression 7.

$${C}_{ij} = {\sum }_{t = 0}^{T} ( {r}_{i , t} \times {r}_{j , t} ) \qquad [ Mathematical expression 7 ]$$

Using this mathematical expression, interactions between brain regions are computed through the product of feature representations r_{i,t} and r_{j,t} of two brain regions according to time t and integrated into a spatiotemporal correlation matrix. The correlation matrix generated in this manner can play an important role in analyzing the spatiotemporal characteristics of brain activity related to ASD.

Therefore, the spatial and temporal convergence feature unit 240 can serve as an important device capable of analyzing spatiotemporal correlations of the brain and providing information on interactions between brain regions essential for ASD diagnosis.

The graph generator 250 may represent the connectivity between regions of interest of the brain in the form of a graph composed of nodes and edges. Based on the correlation matrix derived from the spatial and temporal convergence feature unit 240, the graph generator 250 may represent brain functional connectivity by setting each region of interest of the brain as a node and implementing an interaction between regions of interest as an edge. At this time, the node represents a specific brain region, and the edge serves to reflect the degree of connectivity between brain regions.

In addition, the graph generator 250 may quantify the connectivity between regions of interest of the brain using values derived through the correlation matrix and determine a weight of each edge according to the values. The weight indicates the strength of an interaction between regions of interest and can be analyzed by the GNN to be used to predict presence or absence of ASD.

Specifically, the graph generator 250 may quantify the strength of connection between regions of interest of the brain on the basis of a connectivity matrix C derived from fMRI data and convert the same into an adjacency matrix A of a graph. Each element Cij of the connectivity matrix C indicates the strength of connection between brain regions i and j, and the strength of each edge is determined based on this value. The strength of connection is converted into a discretized edge label on the basis of the quartile. For example, the value Cij can be classified according to each quartile as represented by the following mathematical expression 8.

$$[ Mathematical expression 8 ]$$

$$\{ \begin{matrix}1 & if & {C}_{ij} ∈ {Q}_{1} \\ 2 & if & {C}_{ij} ∈ {Q}_{2} \\ 3 & if & {C}_{ij} ∈ {Q}_{3} \\ 4 & if & {C}_{ij} ∈ {Q}_{4}\end{matrix} , where {Q}_{n} is n - th quartile . \qquad ( 8 )$$

Here, Qn is the n-th quartile, and this classification method discretizes connectivity to make the same suitable for analysis of the GNN.

Accordingly, the graph generator 250 can clearly quantify the correlation between regions of interest and can provide important information in diagnosis of neurodevelopmental disorder such as ASD by indicating a relative importance of the connection strength through the generated edge label.

The graph classifier 260 may analyze the connectivity between regions of interest of the brain on the basis of a graph neural network (GNN) to classify whether there is an ASD. The graph classifier 260 generates graph embedding on the basis of the graph structure derived by the graph generator 250, and the embedding is a method of expressing the connectivity between brain regions as a spatiotemporal pattern.

The graph classifier 260 may transfer the generated graph embedding to a fully connected layer to learn and analyze the characteristics of each connectivity pattern. The graph classifier 260 may finally classify whether there is an ASD using a softmax layer on the basis of the analyzed information. Through this process, the graph classifier 260 can play an important role in diagnosing an ASD by quantifying the correlation between brain regions.

Specifically, the graph classifier 260 receives a graph structure in which each region of interest of the brain is represented as a node and connectivity between regions of interest is represented as an edge, and performs an operation of embedding this graph structure into a high-dimensional space. In this process, graph embedding is performed by reflecting the correlation and connection strength between each node and edge.

The specific processing process of the graph classifier 260 is as follows.

1. Graph embedding generation: A feature vector X^{l}(v) for each node v of the graph is calculated based on the relationship with a neighbor node u. At this time, the connection strength between the nodes is adjusted by a weight

$${Θ}_{uv}^{l}$$

assigned to the edge. The graph embedding process can be defined by the following mathematical expression 9.

$${X}^{l} ( v ) = σ ( {\sum }_{u ∈ N ( v )} {Θ}_{uv}^{l} \cdot {X}^{l - 1} ( u ) ) \qquad [ Mathematical expression 9 ]$$

Here, N(v) represents a neighbor node of node v,

$${Θ}_{uv}^{l}$$

is a weight matrix for an edge, X^{l-1}(u) is the feature vector of node u in the previous layer, and σ is an activation function.

2. Edge weight computation: The weight

$${Θ}_{uv}^{l}$$

of each edge is determined by a learnable function ƒ^{l} according to the label L(u,v) of the edge. Accordingly, the importance of connectivity between nodes is determined by reflecting structural information of the graph, which can be represented as the following mathematical expression 10.

$${Θ}_{uv}^{l} = {f}^{l} ( L ( u , v ) ) \qquad [ Mathematical expression 10 ]$$

This process effectively reflects connectivity within the graph, and plays an important role in analyzing the structural and functional connectivity of the brain related to ASD.

3. Final classification of graph embedding: After graph embedding is completed, the embedded graph feature X^{L} is applied to a classification operation through the fully connected layer FC, and presence or absence of ASD is finally determined by the softmax function. This can be represented as the following mathematical expression 11.

$$Y = softmax ( FC ( {X}^{L} ) ) \qquad [ Mathematical expression 11 ]$$

In this process, the fully connected layer converts embedding of each graph into a classifiable vector, and softmax can probabilistically predict presence or absence of ASD on the basis of this vector.

The graph classifier 260 can play an important role in analyzing neurological patterns based on the spatiotemporal connectivity of the brain through these operations and accurately classifying whether or not there is ASD based thereon.

The controller 270 may manage the overall control operation of the ASD diagnosis apparatus 200 and manage a control flow or a data flow between the preprocessor 210, the spatial feature extractor 220, the temporal feature extractor 230, the spatial and temporal convergence feature unit 240, the graph generator 250, and the graph classifier 260.

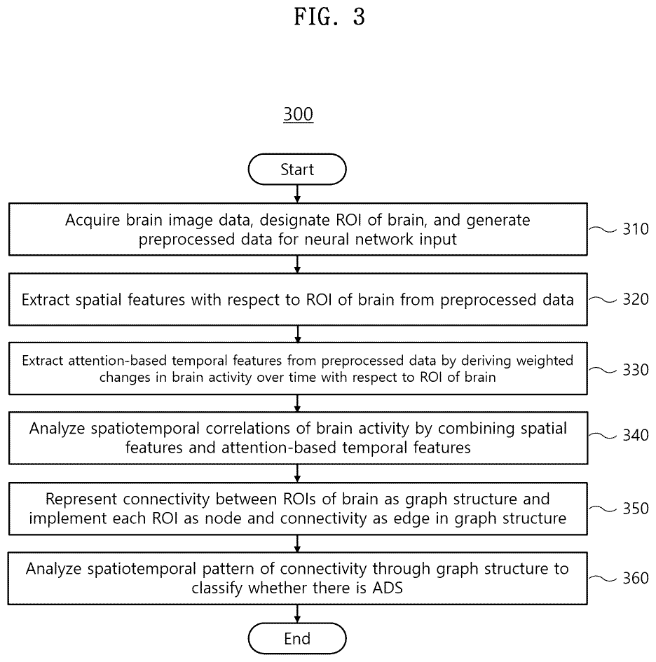

FIG. 3 is a flowchart illustrating the operation of the ASD diagnosis apparatus 200 of FIG. 2.

Referring to FIG. 3, a ASD diagnosis method 300 using a graph neural network performs a preprocessing step 310 of acquiring brain image data and designating a region of interest (ROI) of the brain to generate preprocessed data for neural network input, a spatial feature extraction step 320 of extracting spatial features for a region of interest of the brain from the preprocessed data, a temporal feature extraction step 330 of extracting attention-based temporal features by deriving weighted changes in brain activity over time for a region of interest of the brain from the preprocessed data, a spatial and temporal convergence feature step 340 of analyzing spatiotemporal correlations of brain activity by combining the spatial features and the attention-based temporal features, a graph generation step 350 of representing connectivity between regions of interest of the brain as a graph structure and implementing each region of interest as a node and connectivity as an edge in the graph structure, and a graph classification step 360 of classifying whether or not there is ASD by analyzing spatiotemporal patterns of connectivity through the graph structure.

In the preprocessing step 310, the preprocessor 210 may acquire brain image data and designate a region of interest (ROI) of the brain to generate preprocessed data for neural network input. The preprocessor 210 may perform noise removal and standardization processes using functional brain images such as fMRI, and prepare input data for processing in a neural network.

In the spatial feature extraction step 320, the spatial feature extractor 220 may perform a CNN operation on the basis of preprocessed fMRI data to extract the degree of activation or an activity pattern in a specific region of interest of the brain. The spatial feature extractor 220 may define a regional activation state of the brain through a generated feature map and provide key input data for ASD diagnosis.

In the temporal feature extraction step 330, the temporal feature extractor 230 may input the preprocessed data into a bidirectional LSTM network to derive weighted changes in brain activity over time, and may extract temporal features by applying an attention-based mechanism. The temporal feature extractor 230 may provide temporal data necessary for ASD diagnosis by emphasizing important temporal interactions.

In the spatial and temporal convergence feature step 340, the spatial and temporal convergence feature unit 240 may generate a correlation matrix representing spatiotemporal interactions of brain activity by combining the spatial features and the attention-based temporal features. The spatial and temporal convergence feature unit 240 may analyze spatiotemporal correlations of brain activity through the correlation matrix to derive data for ASD diagnosis.

In the graph generation step 350, the graph generator 250 may generate a graph structure in which each region of interest is a node and connectivity is an edge on the basis of the correlation matrix generated by the spatial and temporal convergence feature unit 240. The graph generator 250 may quantify the connection strength between regions of interest and assign weights to the edges.

In the graph classification step 360, the graph classifier 260 may analyze the generated graph structure to classify whether or not there is an ASD. The graph classifier 260 may generate graph embedding and predict presence or absence of an ASD through a fully connected layer and a softmax layer.

For experiments on the ASD diagnosis method according to the present disclosure, experimental settings were performed on an Ubuntu desktop Linux operating system, and Nvidia DGX Station (Nvidia, Santa Clara, CA, United States) was used as hardware. The Nvidia DGX Station is equipped with 2560 Nvidia tensor cores and includes four Tesla V100 GPUs (each with 64 GB memory) and a 256 GB LRDIMM DDR4 memory.

The software environment was configured in an Ubuntu system with Python 3.x (https://www.python.org/) and TensorFlow version 2.3 (https://www.tensorflow.org/), and various Python libraries (https://pypi.org/) such as Scikit-learn, Nilearn, Nibabel, Monai, and Networkx were utilized during experiments. Through these hardware and software environments, the experiments on the ASD diagnosis method according to the present disclosure were performed efficiently.

The preprocessed ABIDE I and II datasets were used in experiments for ASD diagnosis and were accessible via a web-based platform. Preprocessing was performed using the Configurable Pipeline for the Analysis of Connectomes (C-PAC) and included slice timing and motion correction, intensity normalization (4D global mean=1000), noise signal regression, bandpass filtering (0.01 to 0.1 Hz), and registration to the 3 mm Montreal Neurological Institute (MNI) standard template. All pieces of fMRI data were downsampled to 4 mm in MNI space. This process is described in detail in Table 1.

TABLE 1

| ABIDE I | | | | | | | ABIDE II | | | | | | |
|---|---|---|---|---|---|---|---|---|---|---|---|---|---|
| | Count | | | | Min | Max | | Count | | | | Min | Max |
| Site | ASD | Control | Male | Female | age (y) | age (y) | Site | ASD | Control | Male | Female | age (y) | age (y) |
| CALTECH | 19 | 19 | 30 | 8 | 17.0 | 56.2 | BNI | 29 | 29 | 58 | 0 | 18.0 | 64.0 |
| CMIJ | 14 | 13 | 21 | 6 | 19.0 | 40.0 | EMC | 27 | 27 | 44 | 10 | 6.2 | 10.7 |
| KKI | 22 | 33 | 42 | 13 | 8.07 | 12.77 | ETH | 13 | 24 | 37 | 0 | 13.8 | 30.7 |
| LEUVEN_1 | 14 | 15 | 29 | 0 | 18.0 | 32.0 | GU | 51 | 55 | 71 | 35 | 8.1 | 13.9 |
| LEUVEN_2 | 15 | 20 | 27 | 8 | 12.1 | 16.9 | IP | 22 | 34 | 26 | 30 | 6.1 | 46.6 |
| MAX_MUN | 24 | 33 | 50 | 7 | 7.0 | 58.0 | IU | 20 | 20 | 31 | 9 | 17.0 | 54.0 |
| NYU | 79 | 105 | 147 | 37 | 6.47 | 39.1 | KKI | 56 | 155 | 140 | 71 | 8.0 | 13.0 |
| OHSU | 13 | 15 | 28 | 0 | 8.0 | 15.23 | KUL | 28 | 0 | 28 | 0 | 18.0 | 35.0 |
| OLIN | 20 | 16 | 31 | 5 | 10.0 | 24.0 | NYU_1 | 48 | 30 | 71 | 7 | 5.2 | 34.8 |
| PITT | 30 | 27 | 49 | 8 | 9.33 | 35.2 | NYU_2 | 27 | 0 | 24 | 3 | 5.1 | 8.8 |
| SBL | 15 | 15 | 30 | 0 | 20.0 | 64.0 | OHSU | 37 | 56 | 57 | 36 | 7.0 | 15.0 |
| SDSU | 14 | 22 | 29 | 7 | 8.6 | 17.1 | OILH | 24 | 35 | 40 | 19 | 18.0 | 31.0 |
| STANFORD | 20 | 20 | 32 | 8 | 7.5 | 12.9 | SDSU_1 | 33 | 25 | 49 | 9 | 7.4 | 18.0 |
| TRINITY | 24 | 25 | 49 | 0 | 12.0 | 25.9 | SDSU_2 | 21 | 21 | 38 | 4 | 8.4 | 13.2 |
| UCLA_1 | 49 | 33 | 71 | 11 | 8.4 | 17.9 | TCD | 21 | 21 | 42 | 0 | 10.0 | 20.0 |
| UCLA_2 | 13 | 14 | 25 | 2 | 9.79 | 16.47 | UCD | 18 | 14 | 24 | 8 | 12.0 | 17.8 |
| UM_1 | 55 | 55 | 84 | 26 | 8.2 | 19.2 | UCLA | 16 | 16 | 26 | 6 | 7.8 | 15.0 |
| UM_2 | 13 | 22 | 33 | 2 | 12.8 | 28.8 | USM | 17 | 16 | 28 | 5 | 9.1 | 38.9 |
| USM | 58 | 43 | 101 | 0 | 8.8 | 90 | U_MIA | 13 | 15 | 22 | 6 | 7.1 | 14.3 |
| YALE | 28 | 28 | 40 | 16 | 7.0 | 17.8 | | | | | | | |

ABIDE, Autism Brain Imaging Data Exchange.

Sequence sampling and data augmentation (intensity normalization, random Gaussian noise injection, affine transformation) were applied in the training stage for model training of the ASD diagnosis apparatus according to the present disclosure, and prediction was performed on a continuous sequence composed of 10 frames (window size 10) using the sliding window technique in an inference stage. The final prediction results were aggregated using a soft voting method. The preprocessing pipeline of each piece of data used during experiments was configured as shown in Table 2 below.

TABLE 2

| | | Configure | | |
|---|---|---|---|---|
| Dataset | Type | Train | Valid | Test |
| ABIDE I | ASD | 421 | 47 | 52 |
| | Control | 449 | 50 | 55 |
| ABIDE II | ASD | 422 | 47 | 52 |
| | Control | 481 | 53 | 59 |

For training an ASD diagnosis model, all fMRI images were standardized to a fixed size of 24×24×24 to ensure data consistency. To maintain temporal continuity, the sliding window technique was applied to create a sequence of 10 consecutive frames, allowing the ASD diagnosis model to learn various brain activity patterns. Training and validation data was divided into patient units, with 90% of the data used as a training set and the remaining 10% used as a validation set. The detailed composition of the ABIDE I and II datasets used here and the structural features of the proposed model are shown in Table 3 below.

TABLE 3

| | Time | In | Out | Kernel | | | | | |
|---|---|---|---|---|---|---|---|---|---|
| Operation | Distributed | Channels | Channels | (Layer) | Stride | Padding | BatchNorm | Dropout | Activation |
| Input: | 10 × 1 × 24 × 24 × 24 (1 channel voxel with time) | | | | | | | | |
| Input stem | True | 1 | 32 | 3 × 3 × 3 | 2 × 2 × 2 | 1 × 1 × 1 | True | 0.3 | ReLU |
| Layer 1 | True | 32 | 64 | 3 × 3 × 3 | 1 × 1 × 1 | 1 × 1 × 1 | True | 0.3 | ReLU |
| Layer 2 | True | 64 | 128 | 3 × 3 × 3 | 1 × 1 × 1 | 1 × 1 × 1 | True | 0.3 | ReLU |
| Max pooling | True | 128 | 128 | 2 × 2 × 2 | 2 × 2 × 2 | X | X | X | X |
| Layer 3 | True | 128 | 256 | 3 × 3 × 3 | 1 × 1 × 1 | 1 × 1 × 1 | True | 0.3 | ReLU |
| Layer 4 | True | 256 | 512 | 3 × 3 × 3 | 1 × 1 × 1 | 1 × 1 × 1 | True | 0.3 | ReLU |
| Global | True | — | — | 3 × 3 × 3 | — | — | — | — | — |
| Average Pooling | | | | | | | | | |
| Bi-Directional | True | 512 | 1024 | 2 | — | — | — | X | ReLU |
| LSTM | | | | | | | | | |
| Multi-head | — | 1024 | 1024 | 8 | — | — | — | X | ReLU |
| Attention | | | | | | | | | |
| Fully | — | 1024 | 256 | — | — | — | True | 0.5 | ReLU |
| Connected 1 | | | | | | | | | |
| Fully | — | 256 | 2 | — | — | — | — | 0.3 | Softmax |
| Connected 2 | | | | | | | | | |

| | |
|---|---|
| Optimizer | Adam (lr = 0.0001, weight decay = 0.0005) |
| Batch size | 128 |
| Epochs | 1000 epochs (early stopping 20) |
| Lr scheduler | StepLR (step_size = 50, gamma = 0.5) |
| Total Params: | 20,236,146 |

ReLU, Rectified Linear Unit.

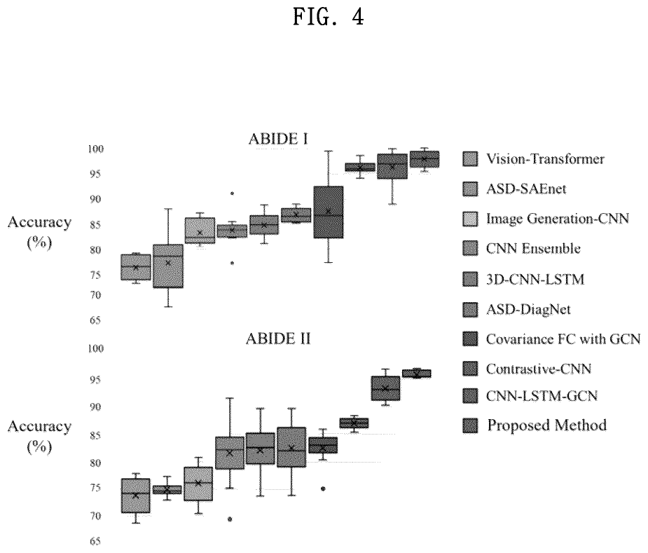

FIG. 4 is a box plot visually comparing the diagnostic accuracy of the ASD diagnosis method according to the present disclosure with those of several existing models.

Details of the ABIDE I and II datasets used in FIG. 4 are presented in Table 4 below.

TABLE 4

| Task (dataset) | Method | Accuracy |
|---|---|---|
| ABIDE I | Bootstrapping ensemble of GCNNs [47] | 68.46 ± 7.94 |
| | ASD-DiagNet [31] | 76.22 ± 2.29 |
| | Multi-view ensemble learning [44] | 74.16 ± 1.31 |
| | ASD-SAEnet [32] | 77.13 ± 5.44 |
| | R-walk [45] | 73.45 ± 2.81 |
| | CNN Ensemble [29] | 83.67 ± 3.24 |
| | 3D-CNN-LSTM [30] | 84.65 ± 2.21 |
| | ResNet-LSTM with self-attention [41] | 86.74 ± 1.34 |
| | Covariance FC with GCN | 87.66 ± 6.84 |
| | Self-supervised ensemble [46] | 94.13 ± 3.12 |
| | Proposed Method | 97.88 ± 1.64 |
| ABIDE II | Multi-view ensemble learning [44] | 73.75 ± 2.97 |
| | ASD-SAEnet [32] | 74.81 ± 1.13 |
| | R-walk [45] | 75.99 ± 3.28 |
| | CNN Ensemble [29] | 81.48 ± 5.66 |
| | 3D-CNN-LSTM [30] | 82.38 ± 4.74 |
| | ResNet-LSTM with self-attention [41] | 82.39 ± 2.93 |
| | Covariance FC with GCN | 86.94 ± 0.91 |
| | Self-supervised ensemble [46] | 93.23 ± 2.18 |
| | Proposed Method | 95.35 ± 0.12 |

As can be seen in Table 4 above, the method according to the present disclosure has performance improvement of about 3.7% p in the ABIDE I dataset and about 2.1% p in the ABIDE II dataset. This performance difference is due to the following two main reasons. Firstly, the existing graph-based approach, the covariance FC method, may not sufficiently capture the neurological characteristics of the brain. On the other hand, the method of the present disclosure can analyze the correlation of fMRI data from various angles through the multi-head attention mechanism and effectively emphasize heterogeneous data characteristics. Secondly, while the existing GCN-based approach utilizes only node features, the method of the present disclosure performs embedding more effectively by using the edge condition layer that models the correlation between nodes, and as a result, it can be confirmed that the method of the present disclosure improves the diagnostic performance.

The ASD diagnosis method (yellow box) according to the present disclosure proposed in FIG. 4 maintains a stable upper accuracy range compared to other existing methods, and in particular, it exhibits superior performance compared to other models in the ABIDE II dataset. This suggests that the ASD diagnosis method according to the present disclosure can provide excellent performance while maintaining high accuracy even in various data environments.

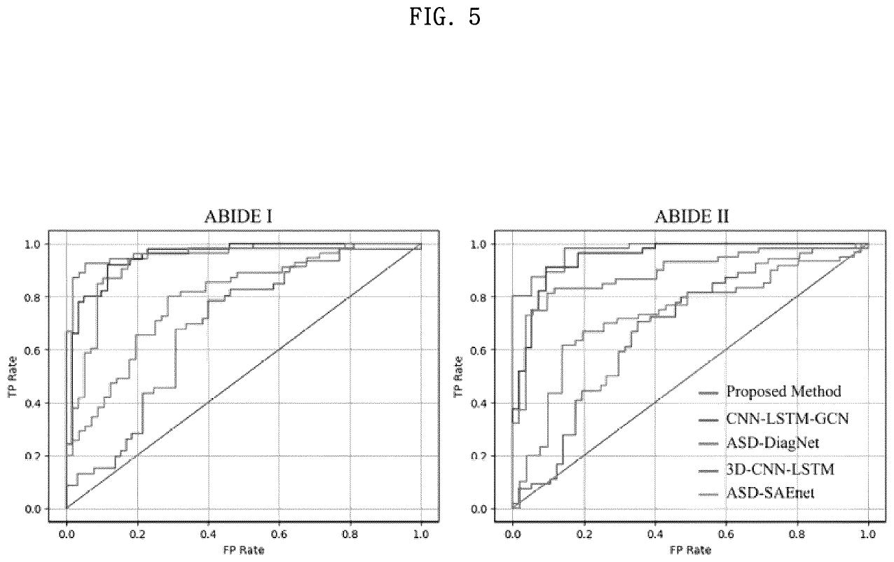

FIG. 5 is a graph showing receiver operating characteristic (ROC) curves evaluating the performance of the ASD diagnosis apparatus according to the present disclosure.

The ROC curve represents the relationship between a true positive rate (TP Rate) and a false positive rate (FP Rate) and is used as an indicator to evaluate the diagnostic accuracy of the model.

In the case of FIG. 5, the red curve represents the ASD diagnosis apparatus according to the present disclosure, and the other curves compare the performances of existing models such as CNN-LSTM-GCN and ASD-DiagNet. The diagnosis apparatus of the present disclosure maintains a high true positive rate (TP Rate) and a low false positive rate (FP Rate), demonstrating superior performance compared to existing models.

In particular, since the area under the ROC curve (AUC) is wider than other models, it is possible to confirm that the diagnosis apparatus of the present disclosure is superior in prediction accuracy. This exhibits that the spatiotemporal correlation of fMRI data is effectively extracted through the multi-head attention mechanism, and high performance is recorded in the ABIDE I and ABIDE II datasets.

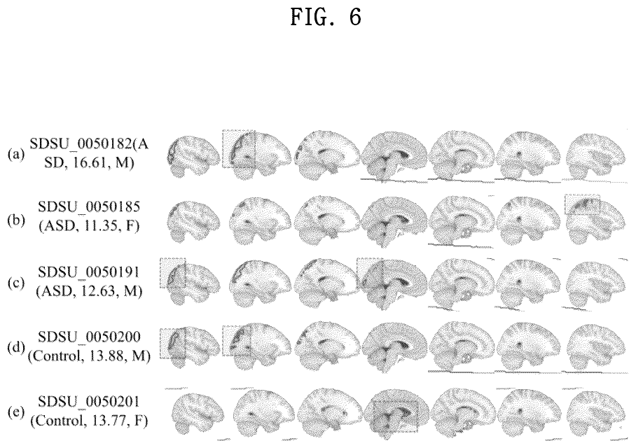

FIG. 6 is a brain scan image visually showing results of activation of a region of interest (ROI) of the brain analyzed by the ASD diagnosis apparatus according to the present disclosure.

FIG. 6 visually shows activated patterns in the region of interest (ROI) of the brain through the ASD diagnosis apparatus according to the present disclosure. FIG. 6 shows a brain activation map of the brain that vary depending on presence or absence of ASD, age, and sex, and (a) to (c) of FIG. 6 show brain scans of ASD patients, and (d) and (e) of FIG. 6 show brain scans of non-ASD patients.

In the case of FIG. 6, the red box in each brain scan image indicates an activation pattern that is commonly observed in both patients with and without the disease, and the blue box indicates an activation pattern that appears similarly regardless of the disease status. The ASD diagnosis apparatus according to the present disclosure can precisely capture the activation of a specific region of the brain through this activation map, and can analyze complex external factors such as age, sex, and disease progression status together. The proposed diagnosis method effectively detects subtle changes in the brain according to various variables during ASD diagnosis, thereby enabling precise diagnosis tailored to characteristics of individual patients.

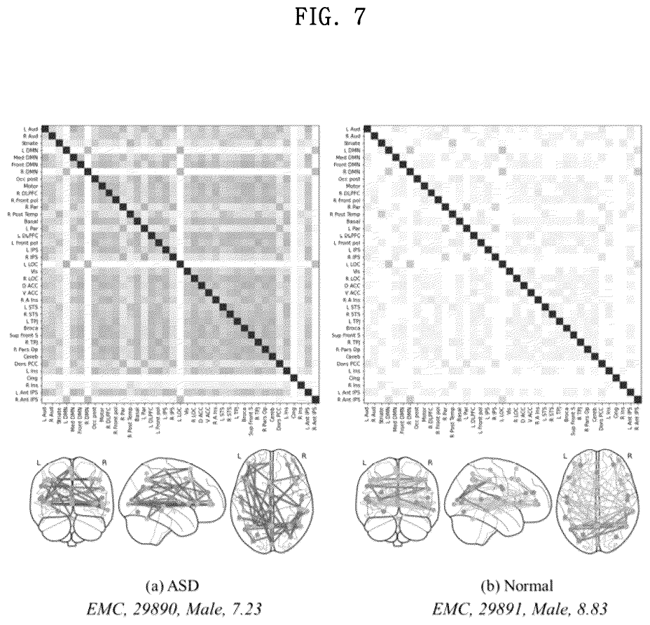

FIG. 7 is a diagram visually showing analysis results of the ASD diagnosis apparatus according to the present disclosure, and shows correlation matrices and brain regional connectivity graphs for comparison of functional connectivities in the brains of an ASD patient and a normal control group.

FIG. 7 is a graph visually representing correlation matrices and connectivity between brain regions extracted by the ASD diagnosis apparatus according to the present disclosure. (a) of FIG. 7 shows a correlation matrix and connectivity graph based on brain data of an ASD patient, and (b) of FIG. 7 shows a correlation matrix and connectivity graph based on brain data of a normal person.

In the case of FIG. 7, a correlation matrix generated using ASD patient data exhibits strong interconnectivity between various brain regions. The ASD diagnosis apparatus effectively reflects the characteristics of ASD by deriving such interconnectivity from time-series features strengthened through an attention mechanism. On the other hand, a correlation matrix based on normal person data exhibits relatively weak connectivity, and the attention mechanism does not form strong connections between specific brain regions, and thus differentiated characteristics are not emphasized.

The ASD diagnosis method according to the present disclosure can precisely capture the neurobiological differences between ASD patients and normal people by combining the attention mechanism and the graph neural network (GNN). This differentiated analysis can play an important role in distinguishing brain patterns with ASD from brain patterns in normal states and ensuring high accuracy in the diagnosis process.

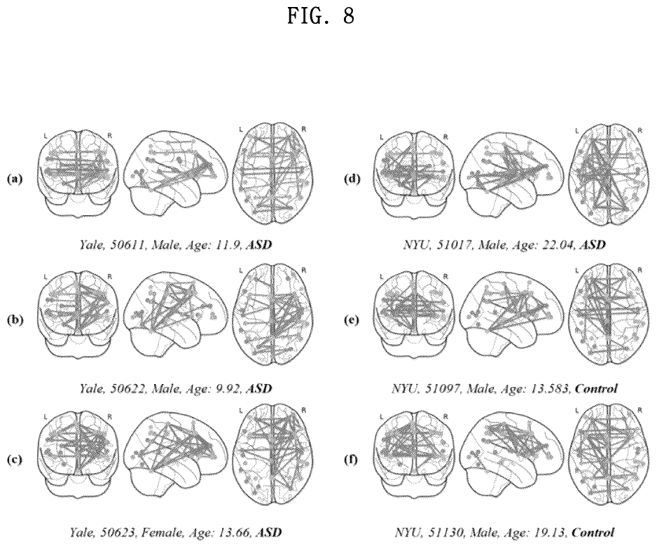

FIG. 8 is a diagram visually comparing brain functional connectivities of an ASD patient and a normal control group as an analysis result of the ASD diagnosis apparatus according to the present disclosure.

FIG. 8 is a graph visually comparing functional connectivity (FC) representing connectivity between brain regions between ASD patients and normal individuals. The institution from which data was collected, subject ID, sex, age, and disease status (presence or absence of ASD) are written below each image, and accordingly, brain connectivity patterns between ASD patients and normal individuals can be compared. (a) to (c) of FIG. 8 show the functional connectivity FC of ASD patients, and (d) to (f) of FIG. 8 show the functional connectivity FC of normal individuals.

Here, in (a) and (b) of FIG. 8, it is possible to ascertain the difference in brain connectivity patterns of ASD patients of different ages, which suggests that the brain activation characteristics of ASD may differ by age. In addition, when (a) of FIG. 8 is compared with (c) of FIG. 8, it is possible to ascertain the difference in brain activation patterns by sex, which indicates the influence of sex on the brain characteristics of ASD. These observations suggest that the brain activation patterns of ASD may differ depending on various factors such as sex and age.

The performance of the proposed method for distinguishing the difference between ASD patients and normal individuals is evaluated by whether it can effectively capture these various differences and contribute to diagnosis. The ASD diagnosis apparatus of the present disclosure using multi-head attention and edge label-based graph neural network (GNN) has strength in accurately distinguishing such subtle differences, and can especially identify the differences well even when the presence or absence of disease is unclear. This analysis emphasizes that the proposed method is effective in capturing complex patterns of brain connectivity and improving the accuracy of diagnosis at the time of diagnosing ASD.

Table 5 below shows results showing that the ASD diagnosis method according to the present disclosure outperforms existing models in detecting ASD and has the potential to contribute to improving early diagnosis and personalized medical strategies.

TABLE 5

| | p-value | Corrected p-value |
|---|---|---|
| Method | (Wilcoxon) | (FDR) |
| **CNN Ensemble | 3.5 × 10⁻⁹ | 2.8 × 10⁻⁸ |
| **Covariance FC with GCN | 6.58 × 10⁻⁸ | 2.6 × 10⁻⁷ |
| **ASD-SAEnet | 2.2 × 10⁻⁶ | 5.9 × 10⁻⁶ |
| **Vision-Transformer | 1.3 × 10⁻⁵ | 52.6 × 10⁻⁵ |
| **3D-CNN-LSTM | 3.4 × 10⁻⁵ | 5.4 × 10⁻⁵ |
| **ASD-DiagNet | 4.1 × 10⁻⁵ | 5.4 × 10⁻⁵ |
| *Contrastive CNN | 1.1 × 10⁻² | 1.3 × 10⁻³ |
| CNN-LSTM-GCN | 8.4 × 10⁻³ | 8.4 × 10⁻³ |

*p < 0.005.
**p < 0.0005.
FDR, false discovery rate.

Table 5 above shows the results of comparing statistical significance between the ASD diagnosis method proposed in the present disclosure and various existing methods. Respective rows represent different models, and columns list p-value derived through the Wilcoxon test and the FDR (false discovery rate) correction value for the p-value.

The statistical analysis results show that the ASD diagnosis method proposed in the present disclosure exhibited significant differences when compared with CNN Ensemble, Covariance FC with GCN, ASD-SAEnet, Vision-Transformer, 3D-CNN-LSTM, ASD-DiagNet, Contrastive CNN, and CNN-LSTM-GCN. For example, p-value compared with CNN Ensemble was very low as 3.5×10⁻⁹, and maintained a significant level of 2.8×10⁻⁸ even after FDR correction. Compared with other models, both the p-value and FDR correction value are low, which suggests that performance differences between the proposed method and existing methods are statistically significant.

These results indicate that the proposed ASD diagnosis method provides higher accuracy and reliability than other existing models in ASD diagnosis and contributes to effectively analyzing complex brain activity patterns by utilizing advanced techniques such as an attention-based feature extraction mechanism. Therefore, the proposed ASD diagnosis method sets a new standard in ASD diagnosis and can make an important contribution to the development of personalized treatment plans and promotion of research in the future.

The ASD diagnosis method according to the present disclosure exhibits promising results in brain network analysis, but there are challenges to be solved to develop a truly comprehensive diagnostic tool. In particular, current research focuses on specific diseases, making it difficult to develop a universal diagnostic model. To overcome this, a standardized preprocessing procedure for diagnosing various neurological conditions and multi-modal data integration are required. In addition, the introduction of a transformer-based model opens up new possibilities, but both computational efficiency and clinical significance need to be considered in order to handle the complexity of fMRI data.

The ASD diagnosis method according to the present disclosure provides a technique for diagnosing ASD with high accuracy by integrating attention-based feature extraction, graph transformation, and a graph neural network (GNN). This method recorded a diagnostic accuracy of 97.88% in ABIDE I datasets and 95.35% in ABIDE II datasets, exhibiting performance improvement of up to 3.7% p compared to the existing SOTA model. This performance increase is due to the ability to more clearly ascertain the neurobiological mechanisms in the process of diagnosing ASD and to effectively distinguish differences from normal brain patterns. Future research will focus on maximizing the synergy effect with graph learning and developing a more precise neural network-based diagnostic model by improving the brain feature extraction technique using contrastive learning.

Although the preferred embodiments of the present disclosure have been described above, those skilled in the art can understand that the present disclosure can be modified and changed in various manners within the scope of the spirit and scope of the present disclosure described in the following claims.

### [Acknowledgement]

Project Serial No: 2710006677
Project No: RS-2020-II201361
Department: Ministry of Science and ICT
Project management (Professional) Institute: Institute of Information & Communications Technology Planning & Evaluation
Research Project Name: Nurturing ICT and Broadcasting Innovation Talents (R&D)
Research task Name: Artificial Intelligence Graduate School Support Project (Yonsei University)
Project Performing Institute: University Industry Foundation, Yonsei University
Research Period: 2024.01.01˜ 2024.12.31

### DETAILED DESCRIPTION OF ELEMENTS

100: Diagram illustrating the process of diagnosing autism spectrum disorder (ASD)
200: Autism spectrum disorder (ASD) diagnosis apparatus
210: Preprocessor
220: Spatial feature extractor
230: Temporal feature extractor
240: Spatial and temporal feature conversion unit
250: Graph generator
260: Graph classifier
270: Controller

1. An apparatus for diagnosing an autism spectrum disorder (ASD) using a graph neural network, the apparatus comprising:
a controller configured to:
acquire brain image data including acquire functional magnetic resonance imaging (fMRI) data:
acquire a region of interest (ROI) of a brain as a plurality of window unit images through a sliding window;
generate preprocessed data for neural network input;
perform a convolutional neural network (CNN) operation on the preprocessed data;
generate a feature map representing a degree of activation and an activity pattern with respect to the ROI as spatial features;
extract attention-based temporal features from the preprocessed data by deriving weighted changes in brain activity over time with respect to the ROI of the brain;
analyze spatiotemporal correlations of brain activity by combining the spatial features and the attention-based temporal features;
represent connectivity between regions of interest of the brain as a graph structure, each of the regions of interest being implemented as a node and the connectivity being implemented as an edge in the graph structure:
analyze a spatiotemporal pattern of the connectivity through the graph structure to classify whether or not there is the ASD; and
provide a brain scan image that visually shows results of activation of the ROI of the brain, analyzed by the apparatus, wherein the brain scan image shows activated patterns in the ROI of the brain and indicates whether or not there is the ASD, and wherein the brain scan image shows brain scans of ASD patients and brain scans of non-ASD patients.

2-3. (canceled)

4. The apparatus of claim 1, wherein the controller is further configured to:
apply a bidirectional long short-term memory (LSTM) network to the preprocessed data; and
generate temporal features of previous or subsequent activities occurring in the ROI.

5. The apparatus of claim 4, the controller is further configured to:
apply a multi-head attention mechanism to the temporal features; and
generate attention representing temporal interactions of the activities.

6. The apparatus of claim 5, wherein the controller is further configured to:
integrate the temporal features and the attention;
performs global average pooling; and
summarize the weighted changes in the brain activity into the attention-based temporal features.

7. The apparatus of claim 1, wherein the controller is further configured to:
generate a correlation matrix representing a degree of spatiotemporal interaction of the brain activity by combining the spatial features and the attention-based temporal features; and
represent the spatiotemporal correlations.

8. The apparatus of claim 7, wherein the controller is further configured to:
quantify the connectivity between the regions of interest of the brain through the correlation matrix; and
determine a weight of the edge.

9. The apparatus of claim 1, wherein the controller is further configured to:
generate graph embedding for the graph structure; and
classify whether or not there is the ASD through a fully connected layer and a softmax layer.

10. A method of diagnosing an autism spectrum disorder (ASD) using a graph neural network, performed in an apparatus for diagnosing the ASD using a graph neural network, the method comprising:
acquiring brain image data including acquire functional magnetic resonance imaging (fMRI) data:
acquiring a region of interest (ROI) of a brain as a plurality of window unit images through a sliding window;
generating preprocessed data for neural network input;
performing a convolutional neural network (CNN) operation on the preprocessed data;
generating a feature map representing a degree of activation and an activity pattern with respect to the ROI as spatial features;
extracting attention-based temporal features from the preprocessed data by deriving weighted changes in brain activity over time with respect to the ROI of the brain;
analyzing spatiotemporal correlations of brain activity by combining the spatial features and the attention-based temporal features;
representing connectivity between regions of interest of the brain as a graph structure, each of the regions of interest being implemented as a node and the connectivity being implemented as an edge in the graph structure;
analyzing a spatiotemporal pattern of the connectivity through the graph structure to classify whether or not there is the ASD; and
providing a brain scan image that visually shows results of activation of the ROI of the brain, analyzed by the apparatus, wherein the brain scan image shows activated patterns in the ROI of the brain and indicates whether or not there is the ASD, and wherein the brain scan image shows brain scans of ASD patients and brain scans of non-ASD patients.
